(19) United States
(12) Patent Application Publication
(10) Pub. No.: US 2022/0395324 A1
(43) Pub. Date: Dec. 15, 2022

(54) METHOD AND DEVICE FOR INTRAMYOCARDIAL INFUSION OF CONDUCTIVE NANOPARTICLES

(71) Applicant: Ethicon, Inc., Somerville, NJ (US)

(72) Inventor: Matthew Kuhn, Houston, TX (US)

(21) Appl. No.: 17/828,394

(22) Filed: May 31, 2022

Related U.S. Application Data
(60) Provisional application No. 63/210,317, filed on Jun. 14, 2021.

(51) Int. Cl. A61B 18/14; A61B 18/24

(52) U.S. Cl. CPC A61B 18/1492; A61B 18/24; A61M 2025/015

(57) ABSTRACT

Disclosed herein are methods to restore myocardial conduction via the injection of conductive nanoparticles into the myocardium via an endovascular injection catheter.

### CROSS-REFERENCE TO RELATED APPLICATIONS

This application claims priority to and the benefit of U.S. Patent Application No. 63/210,317 filed Jun. 14, 2021, which is hereby incorporated by reference in their entirety.

### BACKGROUND

Fundamental biological processes in higher-order organisms rely on conduction and transduction of electrical currents. For example, cognitive, sensory, motor, and visceral functions all rely on efficient conduction of electrical impulses. Traumatic or degenerative injuries that interrupt the connectivity of the physiological circuits often cannot be repaired by natural regeneration or surgical intervention and can have devastating effects.

Myocardial infarction (MI), a potential consequence of myocardial ischemia, signifies cardiac muscle damage where the cells undergo necrosis and apoptosis, eventually forming a permanent scar within the tissues of the heart, such as the ventricular wall. With scar tissue having low electrical conductivity and thus inhibiting the conduction of electrical impulses along normal conduction pathways within tissue, the heart's functionality and ability to pump efficiently are often deteriorated following myocardial infarction, further worsening a patient's cardiovascular health and likely leading to the development of other pathophysiological disorders such as cardiac arrhythmias, congestive heart failure, and/or additional myocardial infarctions. Slow conduction and unidirectional conduction block in portions of the heart can lead to the onset of potentially lethal arrhythmias such as ventricular tachycardia or ventricular fibrillation, the leading causes of sudden death in developed countries. In 2012, the World Health Organization (WHO) estimated that cardiovascular disease (CVD) was the leading cause of death affecting 17.5 million people across the globe. Of these fatalities, 7.4 million were the result of ischemic heart disease.

Current treatment options for supporting healthy cardiovascular function following myocardial infarction have limited efficacy, partially because of the lack of a restorative effect on myocardial conduction velocity. Implantable cardioverter defibrillators respond to ventricular tachycardia by delivering either antitachycardia pacing or a shock but do not improve conduction velocity and cannot prevent ventricular tachycardia initiation. Antiarrhythmic drugs such as amiodarone have significant extracardiac toxicity profiles. Since the therapeutic effects of pharmaceutical compounds that decrease myocardial conduction velocity—such as class I antiarrhythmic drugs—are non-tissue-specific (i.e. their effects are not solely restricted to diseased cardiac tissue), these drugs can induce proarrhythmic effects in healthy tissue. Alternatively, therapies that specifically target abnormal regions of cardiac tissue, such as ablation, often rely on the deliberate destruction of tissue. Extensive ablation and further follow-up procedures must often be performed before arrhythmia noninducibility is achieved, increasing the risk of complications.

Recent attempts to repair myocardial lesions propose the use of stem cell therapy, synthetic or cellularized biopolymers, and gene therapy. The underlying principle of committed myogenic or undifferentiated stem cell transfer is in the regeneration of myocardial tissue when the cell solutions are delivered to an infarct area. The method of transfer for these reparatory cells to the damaged myocardial site may be accomplished by means of intravenous (peripheral) infusion, subcutaneous cytokine injection, coronary sinus infusion, intracoronary infusion, and direct intramyocardial injection using an intracardiac catheter (transendocardial) or through open chest surgery (transepicardial). Despite some promising results, these approaches are all still very preliminary, and some concerns exist about their long-term safety and effects on myocardial structural and electrical remodeling.

Improvements are needed.

### SUMMARY

Disclosed herein are catheters, systems, and methods for infusion of electroconductive material, such as conductive nanoparticles.

One general aspect includes a method for improvement of intramyocardial conduction delay. The method also includes guiding an injection catheter to a target location in a body of a patient; causing an injection needle to deploy; and causing, via the deployed injection needle, delivery of a solution comprising biocompatible, electroconductive material to the target location. Other embodiments of this aspect include corresponding computer systems, apparatus, and computer programs recorded on one or more computer storage devices, each configured to perform the actions of the methods.

One general aspect includes a method for improvement of intramyocardial conduction delay. The method also includes guiding an injection catheter to a target location in a body of a patient; positioning the injection catheter proximate a first injection site, causing an injection needle to deploy, advancing the injection needle into tissue at the first injection site, delivering an electroconductive material to the first injection site, causing an injection needle to retract, positioning the injection catheter proximate at least one other injection site located a desired distance away from the first injection site, causing an injection needle to deploy, advancing the injection needle into tissue at least one other injection site located a desired distance away from the first injection site, and delivering an electroconductive material to at least one other injection site located a desired distance away from the first injection site. Other embodiments of this aspect include corresponding computer systems, apparatus, and computer programs recorded on one or more computer storage devices, each configured to perform the actions of the methods.

One general aspect includes an injection catheter for the improvement of intramyocardial conduction delay. The injection catheter also includes a catheter body may include a flexible tubing having a proximal end and a distal end and at least one lumen disposed through at least a portion of the flexible tubing; a tip section disposed adjacent the distal end of the catheter body, the tip section may include a needle passage extending between a proximal end and a distal end, where the needle passage has a proximal region having a proximal diameter and a distal region having a distal diameter less than the proximal diameter; a needle control handle disposed adjacent the proximal end of the catheter body; an injection needle disposed in at least a portion of the needle passage of the tip section, at least a portion of the catheter body, and at least a portion of the needle control handle, the injection needle having a proximal end coupled to the needle control handle and a distal end disposed within the needle passage, where the injection needle is longitudinally slidable so that the distal end of the injection needle extends beyond the distal end of the tip section upon suitable manipulation of the needle control handle; and a needle stop disposed on a portion of the injection needle, where the needle stop has a distal end that is sized to prevent passage of the portion of the injection needle on which the needle stop is mounted from passing into the distal region of the needle passage thereby limiting a distance that the injection needle is capable of extending beyond the distal end of the tip section. Other embodiments of this aspect include corresponding computer systems, apparatus, and computer programs recorded on one or more computer storage devices, each configured to perform the actions of the methods.

One general aspect includes an injection catheter for the improvement of intramyocardial conduction delay. The injection catheter also includes a catheter body may include a flexible tubing having proximal and distal ends and at least one lumen extending therethrough; a tip section disposed adjacent the distal end of the catheter body; a needle control handle disposed adjacent the proximal end of the catheter body; an injection needle disposed in at least a portion of the tip section, at least a portion of the catheter body, and at least a portion the needle control handle, the injection needle having a proximal end coupled to the needle control handle, where the injection needle is longitudinally slidable so that the distal end of the injection needle extends beyond the distal end of the tip section upon suitable manipulation of the needle control handle; and a fluid reservoir in fluid-communication with the injection needle and configured for delivery of electroconductive material through the injection needle to a target location. Other embodiments of this aspect include corresponding computer systems, apparatus, and computer programs recorded on one or more computer storage devices, each configured to perform the actions of the methods.

### BRIEF DESCRIPTION OF THE DRAWINGS

The following drawings show generally, by way of example, but not by way of limitation, various examples discussed in the present disclosure. In the drawings:

FIG. 1 depicts a heart with carbon nanotube fibers.

FIG. 2 depicts an exemplary injection catheter introduced into a heart.

FIG. 3 depicts an exemplary catheter body.

FIG. 4 depicts an exemplary catheter tip with retracted needle.

FIG. 5 depicts an exemplary catheter tip with extended needle.

FIG. 6A depicts a heart with healthy tissue.

FIG. 6B depicts a heart with diseased tissue.

FIG. 6C depicts a heart with treated tissue.

FIG. 7 depicts a graph showing conduction velocity in normal, diseased, and treated myocardial tissue as measured via pacing and read electrodes.

### DETAILED DESCRIPTION

A therapy to create electrical conductions between tissues could offer a restorative option for electrically excitable tissues, such as the ventricular myocardium. The use case for restoring myocardial conduction velocity is just one of many potential applications. For example, electroconductive connections/pathways could be formed on the surface of the skin (or subcutaneously) to embed biomedical sensors or for identification purposes, on the brain to electrically connect regions of the brain or for sensing purposes (such as measuring brain activity), or within various other tissues of the body for diagnostic and/or therapeutic purposes (such as neuromodulation, modulation of organ function, or pain management to name a few) Recently, carbon nanotube fibers (CNTfs) have been implanted over areas of slowed, severed, or blocked myocardial conduction to effectively restore conduction velocity. CNTfs combine the electrical conductivity of metals with the mechanical strength of polymeric fibers, CNTf interface impedance is superior to metals in vitro, and CNTfs have been shown to be chronically safe and biocompatible in vivo.

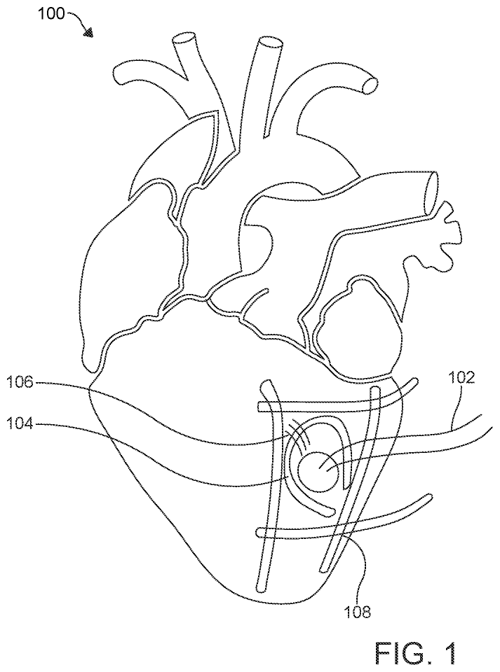

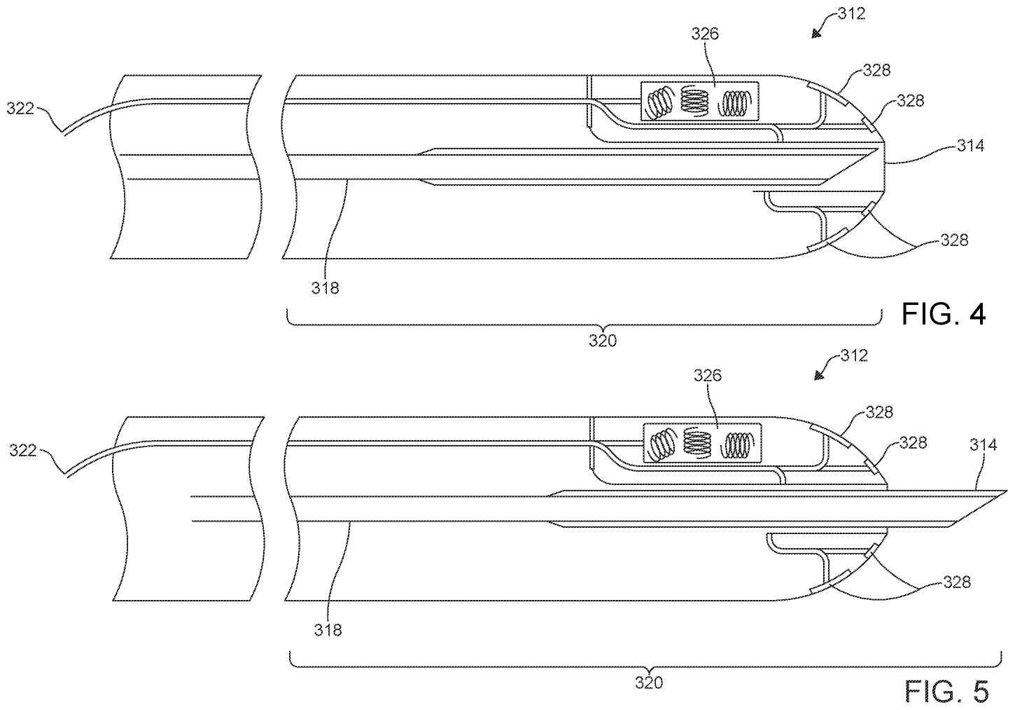

As shown in FIG. 1 below, carbon nanotube fibers (CNTf) 106 were sutured across an area of scar or lesion 104 and were shown to significantly decrease conduction time to near baseline values without controlled external pacing (see also FIG. 4D below). Also depicted are the decapole sensors 108 and the location of the CNT fiber pacing 102. When sewn across the right lateral atrioventricular (AV) junction, CNTfs 106 bridging the AV junction promote antegrade conduction of native sinus rhythm and ventricular preexcitation and promote both antegrade AV conduction and retrograde VA conduction.

While showing promising results, implantation of the CNTfs onto the outer surface of the heart requires highly invasive surgical access, which can lead to complications such as systemic embolization, infection, (possibly chronic) postoperational pain, a prolonged postoperative recovery period, pneumonia, and air leaks. Significantly less invasive catheterization techniques are highly favorable in comparison; however, none yet exist for performing intracardiac suturing. There is a need for a minimally invasive way of restoring conduction velocity to the myocardium.

The present disclosure relates to methods to restore myocardial conduction via the injection of conductive nanoparticles into the myocardium via an endovascular injection catheter.

Disclosed herein are methods for improvement of intramyocardial conduction delay. An example method may comprise guiding an injection catheter to a target location in a body of a patient, causing an injection needle to deploy, and causing, via the deployed injection needle, delivery of a solution comprising biocompatible, conductive nanoparticles to the target location.

An example method may comprise guiding an injection catheter to a target location in a body of a patient, positioning the injection catheter proximate an injection site within the target location, causing an injection needle to deploy, advancing the injection needle into tissue at the injection site, and delivering an electroconductive material to the injection site within the target location.

An example method may comprise guiding an injection catheter to a target location in a body of a patient, causing an injection needle to deploy, and delivering an electroconductive material to the target location.

An example method may comprise guiding an injection catheter to a target location in a body of a patient, positioning the injection catheter proximate a first injection site, causing an injection needle to deploy, advancing the injection needle into tissue at the first injection site, delivering an electroconductive material to the first injection site, causing an injection needle to retract, positioning the injection catheter proximate at least one other injection site located a desired distance away from the first injection site, causing an injection needle to deploy, advancing the injection needle into tissue at least one other injection site located a desired distance away from the first injection site, and delivering an electroconductive material to at least one other injection site located a desired distance away from the first injection site.

Catheters with a needle have been used for injection directly into the myocardium for a variety of treatments, including myocardial revascularization. An endovascular injection catheter may be used to precisely inject conductive nanoparticles into the myocardium to restore myocardial conduction velocity across scar. Such a therapy may allow for the near complete restoration of physiological myocardial conduction velocity across damaged myocardium without the need for surgical intervention.

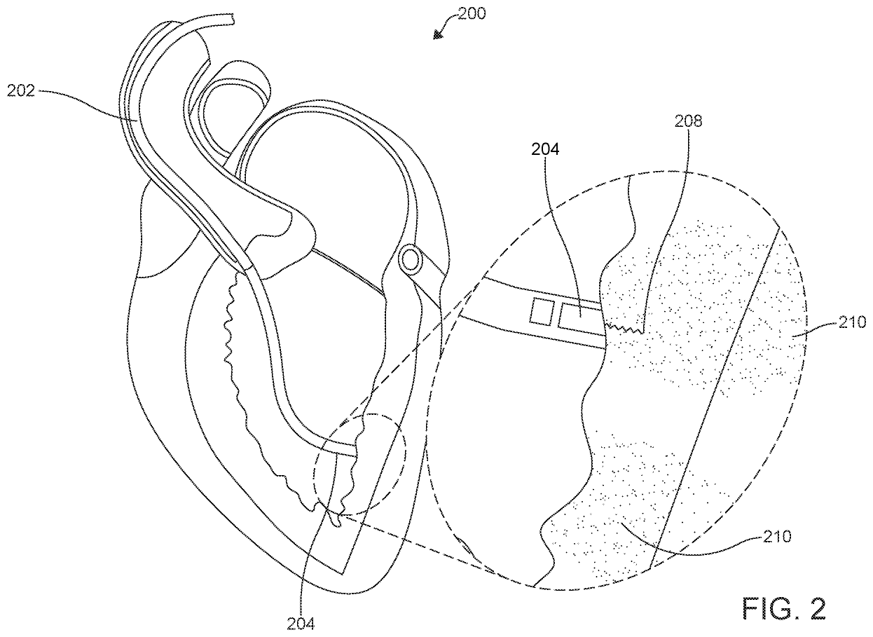

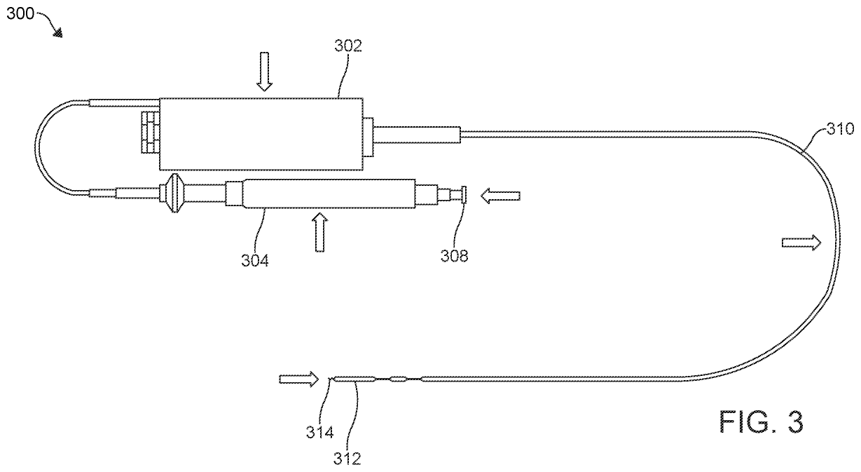

In one embodiment, as depicted in FIG. 3, the present disclosure is directed to a catheter 300 comprising a catheter body comprising a flexible tubing 310 having proximal and distal ends and at least one lumen therethrough. The catheter further includes a tip section 312 comprising a flexible tubing having proximal and distal ends, with the proximal end of the tip section mounted at the distal end of the catheter body. The tip section has a needle passage extending therethough. The needle passage has a proximal region having a proximal diameter and a distal region having a distal diameter less than the proximal diameter. A needle control handle 302 is provided at the proximal end of the catheter body. An injection needle 314 extends through the tip section, catheter body, and needle control handle and has a proximal end attached to the needle control handle 304 and a distal end within the needle passage. The injection needle 314 is longitudinally slidable so that the distal end may extend beyond the distal end of the tip section 312 upon suitable manipulation of the needle control handle 304. A needle stop is mounted on a portion of the injection needle that is positioned within the proximal region of the needle passage. The needle stop has a distal end that is sized to prevent passage of the portion of the injection needle on which the needle top is mounted from passing into the distal region of the needle passage. The needle stop limits the distance that the injection needle may be extended beyond the distal end of the tip section. FIG. 2 below depicts an exemplary injection catheter introduced into the heart.

FIG. 2 depicts a heart 200 with an injection catheter 202 inserted into a major vein or artery and then guided into the chamber 202 of the heart 200 of concern. Navigation of the catheter is accomplished largely with the use of fluoroscopy and electromagnetic position sensors that provide useful data to determine location, orientation, and position of the catheter tip. Injection of therapeutic and diagnostic agents into the myocardium may be imparted via a deployable needle 208 at the catheter tip 204 and a corresponding fluid delivery system located at the needle control handle 308. Affected regions 210 have been injected with the fluid.

FIGS. 3-5 depict an exemplary electrophysiology catheter 300 that is adapted for ablation, mapping, injection, and directional control. In one embodiment, the catheter 300 has a catheter body 302, an intermediate section 310, and a tip section 312 having a tip electrode configured with an omnidirectional distal end and a concentric needle port. The omnidirectional distal end of the tip electrode improves maneuverability and angulation. A dome configuration enables a wide range of tissue contact angles. The concentric needle port provides improved tissue injection success.

FIG. 3 depicts details of the catheter 300. A needle control handle 302 is attached at the proximal end a deployment and retraction mechanism 304 for a spring loaded injection needle 314. This mechanism 304 is connected to a fluid connection 308. The catheter intermediate section or shaft 310 connects the needle control handle 302 distally with the catheter tip 312 and injection needle 314.

FIGS. 4 and 5 show details of the catheter tip. The injection needle 314 is retracted in FIG. 4 and deployed in FIG. 5. In one embodiment, the tip section 320 houses a position sensor 326 arranged in an integrated configuration, wherein the configuration facilitates a path 318 in the tip section 320 for a component, including an injection needle 314, to extend through the tip section 320 for extension and retraction with reduced stress and friction. The integrated configuration is an efficient use of space that allows the tip section 320 to carry both the position sensor 326 for determining location and orientation of the tip section and the contact sensor or sensors 328 (four are depicted in this embodiment). In addition, wiring 322 from these sensors connects to the needle control handle 302. The path 318 defined by the integrated position sensor 326 through the tip section 320 may be generally linear or nonlinear depending on the structure design of the position sensor. For the injection needle 314, the path 318 connects with the concentric needle port whether the path is on axis or off axis with the tip section 320. The catheter also includes a very soft and flexible intermediate section or shaft 310. The catheter tip section 320 may also comprise contact sensors 328 which confirm when the tip section 320 has contacted a body, for instance, the endocardial surface. The injection needle 314 may be off axis or on axis relative to the contact sensor 328.

In one embodiment of the present disclosure, the intermediate section 310 or at least a portion thereof is especially floppy and soft for a “soft touch.” To that end, the intermediate section is constructed of a low durometer tubing reinforced with a spring coil so that stiffness of the intermediate section 310 stems primarily from the components passing therethrough, including the injection needle 314 and its overtubing. The catheter, in an embodiment, includes a needle control handle 302 and a mechanism 304 for the extension and retraction of the needle and also a connection 308 for actuation of fluid flow through the needle. All these actions may be automated such as by means of an automated injection device and system.

In one embodiment of the present disclosure, a solution of biocompatible, conductive nanoparticles is delivered into the myocardium via a series of injections at the endocardial surface. Useful electrical and material properties for an injectable compound to create an interface between two separated areas of electrically-active tissue would include fatigue-resistance, low impedance to ionic charge transfer, biocompatibility, and stability in physiological conditions. Graphene and other conductive nanomaterials may also potentially achieve useful mechanical and electrical properties as implantable materials for electrical bridging of tissues. A therapeutic utility of the intramyocardial injection of conductive nanoparticles includes the improvement of intramyocardial delay to an extent that is sufficient enough to alter the substrate for re-entry.

Disclosed herein are injection catheters. An example injection catheter may comprise a catheter body. The catheter body may comprise a flexible tubing having a proximal end and a distal end and at least one lumen disposed through at least a portion of the flexible tubing, and a tip section disposed adjacent the distal end of the catheter body. The tip section may comprise a needle passage extending between a proximal end and a distal end. The needle passage may have a proximal region having a proximal diameter and a distal region having a distal diameter less than the proximal diameter. A needle control handle may be disposed adjacent the proximal end of the catheter body. An injection needle may be disposed in at least a portion of the needle passage of the tip section, at least a portion of the catheter body, and at least a portion of the needle control handle. The injection needle may have a proximal end coupled to the needle control handle and a distal end disposed within the needle passage. The injection needle may be longitudinally slidable so that the distal end of the injection needle extends beyond the distal end of the tip section upon suitable manipulation of the needle control handle. A needle stop may be disposed on a portion of the injection needle. The injection needle stop may have a distal end that may be sized to prevent passage of the portion of the injection needle on which the needle stop may be mounted from passing into the distal region of the needle passage thereby limiting the distance that the injection needle may be capable of extending beyond the distal end of the tip section.

An example injection catheter may comprise a catheter body. The catheter body may comprise a flexible tubing having proximal and distal ends and at least one lumen extending therethrough. A tip section may be disposed adjacent the distal end of the catheter body. A needle control handle may be disposed adjacent the proximal end of the catheter body. An injection needle may be disposed in at least a portion of the tip section, at least a portion of the catheter body, and at least a portion of the needle control handle. The injection needle may have a proximal end coupled to the needle control handle. The injection needle may be longitudinally slidable so that the distal end of the injection needle extends beyond the distal end of the tip section upon suitable manipulation of the needle control handle. The injection catheter may comprise a fluid reservoir in fluid-communication with the injection needle and configured for the delivery of electroconductive material through the injection needle to a target location.

An example injection catheter may comprise a catheter body. The catheter body may comprise a flexible tubing having proximal and distal ends and at least one lumen extending therethrough. A tip section may comprise a flexible tubing having proximal and distal ends. The proximal end of the tip section may be mounted at the distal end of the catheter body. One or more electrodes may be mounted on the tip section. One or more electrode lead wires may correspond in number to the number of electrodes. Each electrode lead wire may comprise a distal end that may be electrically connected to a corresponding electrode on the tip section. A needle control handle may be disposed at the proximal end of the catheter body. An injection needle may extend through the tip section, catheter body, and needle control handle and have a proximal end attached to the needle control handle and a distal end within the tip section. The injection needle may be longitudinally slidable within the tip section so that the distal end of the injection needle can extend beyond the distal end of the tip section upon suitable manipulation of the needle control handle. The injection needle may consist of plastic tubing having at least a distal region with straight position memory, and a protective tubing extending through the tip section and catheter body through which the needle extends coaxially.

An example injection catheter may comprise a catheter body. The catheter body may comprise a flexible tubing having proximal and distal ends and at least one lumen extending therethrough. The catheter body may comprise a tip section further comprising a flexible tubing having proximal and distal ends. The proximal end of the tip section may be mounted at the distal end of the catheter body. One or more electrodes may be mounted on the tip section, one or more electrode lead wires that correspond in number to the number of electrodes, each electrode lead wire may comprise a distal end that may be electrically connected to a corresponding electrode on the tip section. The injection catheter may comprise a needle control handle proximal the catheter body. The injection catheter may comprise an injection needle extending through the tip section, catheter body, and needle control handle and may comprise a proximal end attached to the needle control handle and a distal end within the tip section. The injection needle may be longitudinally slidable within the tip section so that the distal end of the injection needle can extend beyond the distal end of the tip section upon suitable manipulation of the needle control handle. The injection needle may comprise plastic tubing having at least a distal region with straight position memory. The injection needle may further comprise a piece of metal tubing attached at its end to an end of the plastic tubing and a protective tubing through which the needle extends coaxially.

An example injection catheter may comprise a catheter body. The catheter body may comprise a flexible tubing having proximal and distal ends and at least one lumen extending therethrough, a tip section comprising a flexible tubing having proximal and distal ends. The proximal end of the tip section may be mounted at the distal end of the catheter body. One or more electrodes may be mounted on the tip section, one or more electrode lead wires that correspond in number to the number of electrodes, each electrode lead wire may comprise a distal end that may be electrically connected to a corresponding electrode on the tip section. A needle control handle may be disposed proximal the catheter body. An injection needle may extend through the tip section, catheter body, and needle control handle and have a proximal end attached to the needle control handle and a distal end within the tip section. The injection needle may be longitudinally slidable within the tip section so that the distal end of the injection needle can extend beyond the distal end of the tip section upon suitable manipulation of the needle control handle. The injection needle may comprise plastic tubing having at least a distal region with straight position memory, a protective tubing through which the needle extends coaxially, and a deflection control handle proximal of the catheter body and distal of the needle control handle. The injection needle and protective tube may extend through a guide tube thereby allowing the injection needle and protective tube longitudinal movement within the deflection control handle. The deflection control handle may have a piston for manipulating a puller wire. The injection needle, protective tube and guide tube may extend through a second tunnel in the deflection control handle and a space may be provided between a proximal end of the piston and a distal end of the second tunnel to avoid undesirable bending of the injection needle.

An example injection catheter may comprise a catheter body. The catheter body may comprise an outer wall, proximal and distal ends, and at least one lumen extending therethrough, a tip section further comprising flexible tubing and having proximal and distal ends, and at least one lumen extending therethrough. The proximal end of the tip section may be fixedly attached to the distal end of the catheter body. A tip electrode may be mounted at the distal end of the tip section. The tip electrode may comprise a distal face and a lumen extending therethrough. The injection catheter may comprise an injection needle which may be straight and smooth over its entire length and may extend through the at least one lumen of the catheter body, then extending through the at least one lumen of the tip section and into the lumen of the tip electrode. The injection needle may be in fluid tight engagement with the lumen of the tip electrode. The injection needle may be movable between a first position in which the needle may be contained within the tip electrode, to a second position in which the needle extends out of the distal face of the tip electrode. The injection catheter may comprise a deflection control handle proximal the catheter body. The deflection control handle may comprise a thumb control. The entire thumb control may be longitudinally movable relative to the deflection control handle. The injection catheter may further comprise a needle control handle proximal the deflection control handle and connected to the injection needle for moving the injection needle longitudinally relative to the catheter body.

An example injection catheter may comprise a catheter body. The catheter body may comprise an outer wall, proximal and distal ends and at least one lumen extending therethrough, a tip section further comprising flexible tubing having proximal and distal ends and at least one lumen having an inner diameter extending therethrough, the proximal end of the tip section may be fixedly attached to the distal end of the catheter body. The injection catheter may comprise an injection needle having proximal and distal ends and an outer diameter extending through the at least one lumen in the catheter body, further extending through the at least one lumen of the tip section, the injection needle may be in fluid tight engagement with the at least one lumen of the tip section through which it extends. The injection needle may be slidable between a retracted position, in which the injection needle may be contained within the tip section, and an extended position, in which the injection needle extends out of the distal end of the tip section. The injection needle may comprise a deflection control handle having proximal and distal ends mounted on the proximal end of the catheter body and a thumb control. The entire thumb control may be longitudinally moveable relative to the deflection control handle. The thumb control may be mounted on the deflection control handle. The injection needle may comprise a first puller wire having proximal and distal ends extending through the deflection control handle and the catheter body. The proximal end of the puller wire may be anchored in the deflection control handle and the distal end of the puller wire may be anchored to the tip section, and a needle control handle including a piston.

An example injection catheter may comprise a catheter body. The catheter body may comprise an outer wall, proximal and distal ends and at least one lumen extending therethrough, and a tip section further comprising flexible tubing and having proximal and distal ends and at least one lumen extending therethrough. The proximal end of the tip section may be fixedly attached to the distal end of the catheter body. The injection catheter may further comprise a tip electrode mounted on the distal end of the tip section. The tip electrode may comprise a distal face and a lumen extending therethrough. The injection catheter may comprise an injection needle may comprise proximal and distal ends extending through the at least one lumen of the tip section into the lumen of the tip electrode. The injection needle may be in fluid tight engagement with the lumen of the tip electrode. The injection catheter may be slidable between a retracted position, in which the injection needle may be contained within the tip section, and an extended position, in which the injection needle extends out of the distal face of the tip electrode. The injection catheter may comprise a deflection control handle may comprise a thumb control. The entire thumb control may be longitudinally slidable relative to the deflection control handle. The injection catheter may comprise a puller wire further comprising proximal and distal ends extending through the deflection control handle and the catheter body. The proximal end of the puller wire may be anchored in the deflection control handle, and the distal end of the puller wire may be anchored to the tip section. The injection catheter may comprise a needle control handle further comprising a piston.

An example injection catheter may comprise a catheter body. The catheter body may comprise a flexible tubing having proximal and distal ends and at least one lumen extending therethrough, and a tip section further comprising a flexible tubing having proximal and distal ends. The proximal end of the tip section may be mounted at the distal end of the catheter body. The injection catheter may comprise a tip electrode mounted on the distal end of the tip section, and a needle control handle disposed at the proximal end of the catheter body. The injection catheter may comprise an injection needle comprising an elongated tubing extending through the catheter body, tip section and needle control handle and having a proximal end attached to the needle control handle. The injection needle may also have a distal region within the tip section and an open distal end. The injection needle may be longitudinally slidable within the catheter body so that its distal region can extend beyond the distal end of the tip section upon suitable manipulation of the needle control handle. The distal region of the injection needle may have a plurality of (e.g., four) fluid ports along its length in addition to the open distal end of the needle, wherein a first pair of fluid ports may be positioned on one side of the injection needle and a second-pair of fluid ports may be positioned on an opposite side of the injection needle, wherein each pair of fluid ports includes a distal-most fluid port and a proximal-most fluid port, the distal-most fluid port of each pair of fluid ports may be positioned about two inches from the distal end of the injection needle, and the proximal-most fluid port of each pair of fluid ports may be distanced about 0.02 inches from its corresponding distal-most fluid port, a protective tube mounted coaxially around the injection needle, and an electromagnetic location sensor mounted in the distal end of the tip section, whereby, in use, fluid passes out of the needle through the open distal end of the needle and through the fluid ports along the length of the distal region.

An example injection catheter may comprise a catheter body. The catheter body may comprise an outer wall, proximal and distal ends and at least one lumen extending therethrough, and a tip section may comprise a flexible tubing having proximal and distal ends. The proximal end of the tip section may be mounted at the distal end of the catheter body, a needle control handle at the proximal end of the catheter body. The injection catheter may comprise an injection needle may comprise elongated tubing extending through the tip section, catheter body, and needle control handle and having a proximal end attached to the needle control handle, the injection needle also having a distal region within the tip section and an open distal end. The injection needle may be longitudinally slidable within the tip section so that its distal region can extend beyond the distal end of the tip section upon suitable manipulation of the needle control handle. The distal region of the injection needle may have a plurality of (e.g., four) fluid ports along its length in addition to the open distal end of the needle. A first pair of fluid ports may be positioned on one side of the injection needle and a second-pair of fluid ports may be positioned on an opposite side of the injection needle, wherein each pair of fluid ports includes a distal-most fluid port and a proximal-most fluid port, a protective tube mounted coaxially around the injection needle, whereby, in use, fluid passes out of the needle through the open distal end of the needle and through the fluid ports along the length of the distal region.

An example injection catheter may comprise a catheter body. The catheter body may comprise flexible tubing having proximal and distal ends, a stiffening tube disposed within the flexible tubing, and at least one lumen extending therethrough. The injection catheter may comprise a needle control handle at the proximal end of the catheter body. The needle control handle may comprise an outer body having a piston chamber therein, a piston slidably mounted within the piston chamber, and a compression spring in the piston chamber between the outer body and the piston. The injection catheter may comprise an injection needle extending through the catheter body and at least a portion of the needle control handle and having a proximal end attached to one of the outer body and the piston and a distal end within the catheter body, whereby longitudinal movement of one of the outer body and the piston relative to the other of the outer body and the piston may compress the compression spring and move the injection needle distally relative to the catheter body so that the distal end of the injection needle extends outside the distal end of the catheter body.

An example injection catheter may comprise a catheter body comprising an outer wall, proximal and distal ends, and at least one lumen extending therethrough, a control handle fixedly attached to the proximal end of the catheter body, and a tip section comprising flexible tubing having proximal and distal ends and at least one lumen extending therethrough. The proximal end of the tip section may be fixedly attached to the distal end of the catheter body. The injection catheter may comprise an injection needle which may be straight and smooth over its entire length may extend through the lumen in the catheter body and extending in fluid-tight engagement through the lumen in the tip section, said needle may be slidable from a first position in which the needle may be withdrawn into the tip section to a second position in which the needle extends out of the tip section. The injection catheter may comprise a slidable needle control knob mounted on the control handle and connected to the proximal end of the injection needle for sliding the injection needle from the first position to the second position, and a deflection control mounted on the control handle for deflecting the catheter upon manipulation of the deflection control.

An example injection catheter may comprise a catheter body, an intermediate section distal of the catheter body, and a tip section distal of the intermediate section. The tip section comprising a tip electrode with an omnidirectional distal end and a concentric needle port. The injection catheter may comprise an integrated magnetic device and position sensor, at least one of which may comprise a hollow configuration to receive at least a portion of the other. The injection catheter may comprise a transition section between the intermediate section and the tip section having one or more additional magnetic devices, a needle extending through the catheter body, the intermediate section and the tip section, and a needle control handle adapted to extend and retract the injection needle through the concentric needle port.

The target location may comprise the myocardium. The target location may comprise an area at or adjacent the endocardial surface of the heart. Other locations may be used.

The electroconductive material may comprise a plurality of electrically conductive nanoparticles. The electroconductive material may comprise an aqueous dispersion of metallic nanoparticles. The electroconductive material may comprise a plurality of metallic nanoparticles.

The plurality of electrically conductive nanoparticles may comprise at least one biocompatible metal such as gold, platinum, palladium, nickel, copper, zinc, or silver.

The average diameter of the electrically conductive nanoparticles may be less than 150 nm. Other sizes may be used.

The electroconductive material may comprise a plurality of electrically conductive nanoparticles having a coating on a surface thereof. The coating prevents the particles from agglomerating when the particles are in solution and allows adjacent particle surfaces to be in direct physical contact when the particles are not in solution.

The electroconductive material may comprise a plurality of substantially non-agglomerated metal nanoparticles. The plurality of substantially non-agglomerated nanoparticles may have an electrical resistivity of 1 Ω-cm or less.

The electroconductive material may comprise a plurality of electrically conductive polymer molecules. The average diameter of the electrically conductive polymer molecules may be less than 150 nm.

The electrically conductive polymer may comprise polyvinylpyrrolidone.

The electroconductive material may comprise a conductive choline-based bio-ionic liquid (Bio-IL) conjugated hydrogel. Bio-IL conjugated hydrogels exhibit a wide range of highly tunable physical properties, remarkable in vitro and in vivo biocompatibility, and high electrical conductivity without the need for additional conductive components. The physical properties of ECHs, such as their mechanical properties, water uptake capability, porosity, and degradation rate can influence tissue regeneration in vivo. The electroconductive material may comprise a fluid suspension of carbon nanotube particles. The electroconductive material may comprise a fluid suspension of graphene particles.

An example injection catheter for the improvement of intramyocardial conduction delay may comprise a catheter body. The catheter body may comprise a flexible tubing having a proximal end and a distal end and at least one lumen disposed through at least a portion of the flexible tubing. A tip section may be disposed adjacent the distal end of the catheter body. The tip section may comprise a needle passage extending between a proximal end and a distal end. The needle passage may have a proximal region having a proximal diameter and a distal region having a distal diameter less than the proximal diameter. A needle control handle may be disposed adjacent the proximal end of the catheter body. An injection needle may be disposed in at least a portion of the needle passage of the tip section, at least a portion of the catheter body, and at least a portion of the needle control handle. The injection needle may have a proximal end coupled to the needle control handle and a distal end disposed within the needle passage. The injection needle may be longitudinally slidable so that the distal end of the injection needle extends beyond the distal end of the tip section upon suitable manipulation of the needle control handle. A needle stop may be disposed on a portion of the injection needle. The needle stop may have a distal end that may be sized to prevent passage of the portion of the injection needle on which the needle stop may be mounted from passing into the distal region of the needle passage thereby limiting the distance that the injection needle may be capable of extending beyond the distal end of the tip section.

The injection catheter may further comprise a thumb control mounted on the needle control handle for deflecting the catheter upon manipulation of the thumb control.

The injection catheter may further comprise one or more position sensors disposed in or adjacent the tip section and configured to determine a position of the tip section. The injection catheter may further comprise one or more position sensors disposed in or adjacent the tip section and configured to determine an orientation of the tip section. The injection catheter may further comprise one or more contact sensors disposed in or adjacent the tip section and configured to determine contact of the tip section to a surface.

The distal end of the tip section may have an omnidirectional configuration. The distal end of the tip section may have a dome shape.

The needle port may be disposed on the distal end of the tip section and may be configured to allow the needle to extend outside the distal end of the tip section. The needle port may be disposed concentrically to the distal end of the tip section.

The catheter body may comprise an intermediate section disposed between the tip section and the needle control handle.

The intermediate section may comprise a low durometer tubing reinforced with a spring coil.

An example injection catheter for the improvement of intramyocardial conduction delay may comprise a catheter body comprising a flexible tubing having proximal and distal ends and at least one lumen extending therethrough, a tip section disposed adjacent the distal end of the catheter body, a needle control handle disposed adjacent the proximal end of the catheter body, and an injection needle disposed in at least a portion of the tip section, at least a portion of the catheter body, and at least a portion of the needle control handle. The injection needle may comprise a proximal end coupled to the needle control handle. The injection needle may be longitudinally slidable so that the distal end of the injection needle extends beyond the distal end of the tip section upon suitable manipulation of the needle control handle. The injection catheter may also comprise a fluid reservoir in fluid-communication with the injection needle and configured for the delivery of electroconductive material through the injection needle to a target location.

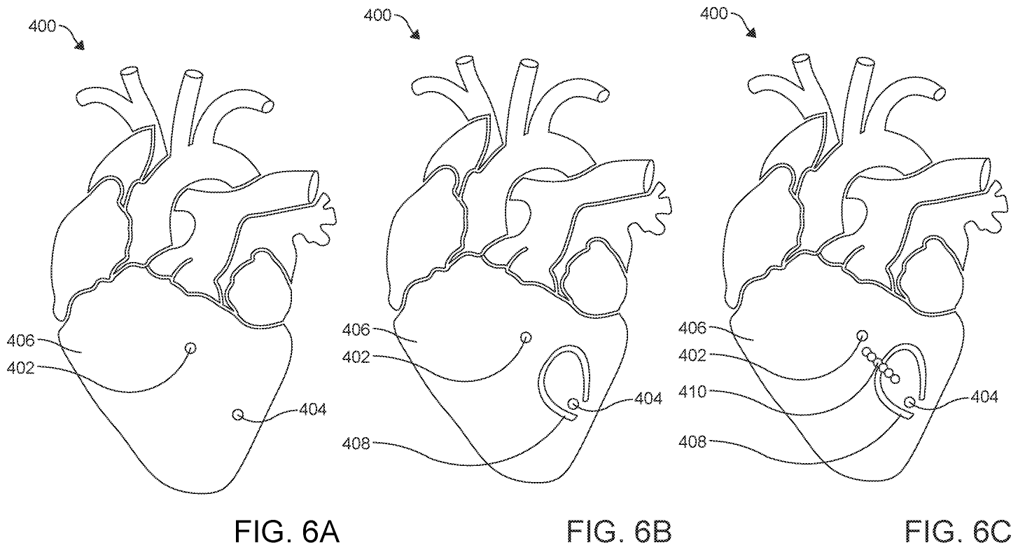

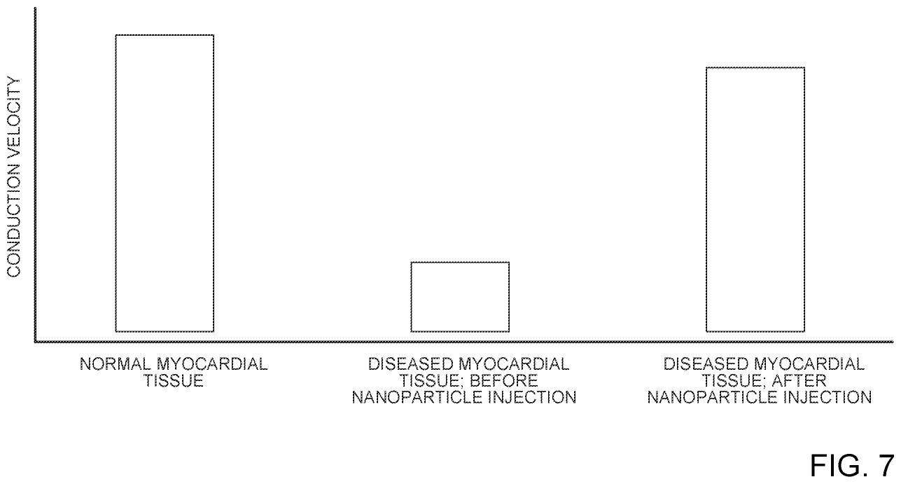

As shown in FIGS. 6A-6C and 7 below, local ischemia arising from myocardial infarction results in the formation of areas of scar tissue 408. With scar tissue 408 having low electrical conductivity and thus being unable to contract, the heart's functionality and ability to pump efficiently are deteriorated, likely leading to congestive heart failure. Slow conduction and unidirectional conduction block in portions of the heart may lead to the onset of lethal arrhythmias known as ventricular tachycardia or ventricular fibrillation.

FIG. 6A shows a healthy heart 400 with no scar tissue. Healthy tissue 406 is shown throughout the heart. A pacing electrode 402 and a read electrode 404 are also depicted. The conduction velocity is normal. FIG. 6B shows a heart 430 with scarred tissue 408. FIG. 6C shows a heart 460 with scarred tissue 408 and also conductive nanoparticle injection sites 410 which bridge over the scarred tissue 408.

FIG. 7 depicts the conduction velocity for normal myocardial tissue, for diseased myocardial tissue before nanoparticle injection (middle) and for diseased myocardial tissue after nanoparticle injection.

### EXAMPLES

Example 1: A catheter comprising: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a needle control handle disposed adjacent the proximal end of the catheter body; an injection needle disposed in at least a portion of the needle passage of the tip section, at least a portion the catheter body, and at least a portion the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 2: The catheter of example 1, further comprising one or more position sensors disposed in or adjacent the tip section and configured to determine a positon of the tip section.

Example 3: The catheter of any one of examples 1-2, further comprising one or more position sensors disposed in or adjacent the tip section and configured to determine an orientation of the tip section.

Example 4: The catheter of any one of examples 1-3, further comprising one or more contact sensors disposed in or adjacent the tip section and configured to determine contact of the tip section to a surface.

Example 5: The catheter of any one of examples 1-4, wherein the distal end of the tip section has an omnidirectional configuration.

Example 6: The catheter of any of examples 1-5, wherein the distal end of the tip section has a dome shape.

Example 7: The catheter of any of examples 1-6, wherein a needle port is disposed on the distal end of the tip section and is configured to allow the needle to extend outside the distal end of the tip section.

Example 8: The catheter of any one of examples 1-7, wherein the needle port is disposed concentrically to the distal end of the tip section.

Example 9: The catheter of any one of examples 1-8, the catheter body comprises an intermediate section disposed between the tip section and the needle control handle.

Example 10: The catheter of any one of examples 1-9, wherein the intermediate section comprises a flexible tubing reinforced with a spring coil.

Example 11: A method for improvement of intramyocardial delay, the method comprising: guiding a needle catheter to a target location in a body of a patient; causing an injection needle to deploy; and causing, via the deployed injection needle, delivery of a solution comprising biocompatible, conductive nanoparticles to the target location.

Example 12: The method of example 11, wherein the target location comprises the myocardium.

Example 13: The method of any one of examples 11-12, wherein the target location comprises an area at or adjacent the endocardial surface of the heart.

Example 14: The method of any one of examples 11-13, wherein the needle catheter comprises: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a needle control handle disposed adjacent the proximal end of the catheter body; the injection needle disposed in at least a portion of the needle passage of the tip section, at least a portion the catheter body, and at least a portion the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 15: A method for improvement of intramyocardial conduction delay comprising: guiding an injection catheter to a target location in a body of a patient; causing an injection needle to deploy; and causing, via the deployed injection needle, delivery of a solution comprising biocompatible, conductive nanoparticles to the target location.

Example 16: A method for improvement of intramyocardial conduction delay comprising: guiding an injection catheter to a target location in a body of a patient; positioning the injection catheter proximate an injection site within the target location; causing an injection needle to deploy; advancing the injection needle into tissue at the injection site; and delivering an electroconductive material to the injection site within the target location.

Example 17: A method for improvement of intramyocardial conduction delay comprising: guiding an injection catheter to a target location in a body of a patient; causing an injection needle to deploy; and delivering an electroconductive material to the target location.

Example 18: A method for improvement of intramyocardial conduction delay comprising: guiding an injection catheter to a target location in a body of a patient; positioning the injection catheter proximate a first injection site; causing an injection needle to deploy; advancing the injection needle into tissue at the first injection site; delivering an electroconductive material to the first injection site; causing an injection needle to retract; positioning the injection catheter proximate at least one other injection site located a desired distance away from the first injection site; causing an injection needle to deploy; advancing the injection needle into tissue at least one other injection site located a desired distance away from the first injection site; and delivering an electroconductive material to at least one other injection site located a desired distance away from the first injection site.

Example 19: The method of any one of Examples 15-18, wherein the injection catheter comprises: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a needle control handle disposed adjacent the proximal end of the catheter body; an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 20: The method of any one of Examples 15-18, wherein the injection catheter comprises: a catheter body comprising a flexible tubing having proximal and distal ends and at least one lumen extending therethrough; a tip section disposed adjacent the distal end of the catheter body; a needle control handle disposed adjacent the proximal end of the catheter body; an injection needle disposed in at least a portion of the tip section, at least a portion of the catheter body, and at least a portion of the needle control handle, the injection needle having a proximal end coupled to the needle control handle, wherein the injection needle is longitudinally slidable so that the distal end of the injection needle extends beyond the distal end of the tip section upon suitable manipulation of the needle control handle; and a fluid reservoir in fluid-communication with the injection needle and configured for the delivery of electroconductive material through the injection needle to a target location.

Example 21: The method of any one of Examples 15-18, wherein the injection catheter comprises: a catheter body comprising a flexible tubing having proximal and distal ends and at least one lumen extending therethrough; a tip section comprising a flexible tubing having proximal and distal ends, wherein the proximal end of the tip section is mounted at the distal end of the catheter body; one or more electrodes mounted on the tip section; one or more electrode lead wires that correspond in number to the number of electrodes, each electrode lead wire having a distal end that is electrically connected to a corresponding electrode on the tip section; a needle control handle at the proximal end of the catheter body; an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle consists of plastic tubing having at least a distal region with straight position memory; and a protective tubing extending through the tip section and catheter body through which the needle extends coaxially.

Example 22: The method of any one of Examples 15-18, wherein the injection catheter comprises: a catheter body comprising a flexible tubing having proximal and distal ends and at least one lumen extending therethrough; a tip section comprising a flexible tubing having proximal and distal ends, wherein the proximal end of the tip section is mounted at the distal end of the catheter body; one or more electrodes mounted on the tip section; one or more electrode lead wires that correspond in number to the number of electrodes, each electrode lead wire having a distal end that is electrically connected to a corresponding electrode on the tip section; a needle control handle proximal the catheter body; an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle comprises plastic tubing having at least a distal region with straight position memory, the injection needle further comprising a piece of metal tubing attached at its end to an end of the plastic tubing; and a protective tubing through which the needle extends coaxially.

Example 23: The method of any one of Examples 15-18, wherein the injection catheter comprises: a catheter body comprising a flexible tubing having proximal and distal ends and at least one lumen extending therethrough; a tip section comprising a flexible tubing having proximal and distal ends, wherein the proximal end of the tip section is mounted at the distal end of the catheter body; one or more electrodes mounted on the tip section; one or more electrode lead wires that correspond in number to the number of electrodes, each electrode lead wire having a distal end that is electrically connected to a corresponding electrode on the tip section; a needle control handle proximal the catheter body; an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle comprises plastic tubing having at least a distal region with straight position memory; a protective tubing through which the needle extends coaxially; and a deflection control handle proximal of the catheter body and distal of the needle control handle, wherein the injection needle and protective tube extend through a guide tube thereby allowing the injection needle and protective tube longitudinal movement within the deflection control handle, the deflection control handle having a piston for manipulating a puller wire, wherein the injection needle, protective tube and guide tube extend through a second tunnel in the deflection control handle and a space is provided between a proximal end of the piston and a distal end of the second tunnel to avoid undesirable bending of the injection needle.

Example 24: The method of any one of Examples 15-18, wherein the injection catheter comprises: a catheter body having an outer wall, proximal and distal ends, and at least one lumen extending therethrough; a tip section comprising flexible tubing and having proximal and distal ends and at least one lumen extending therethrough, the proximal end of the tip section being fixedly attached to the distal end of the catheter body; a tip electrode mounted at the distal end of the tip section, the tip electrode having a distal face and a lumen extending therethrough; an injection needle which is straight and smooth over its entire length extending through the at least one lumen of the catheter body, then extending through the at least one lumen of the tip section and into the lumen of the tip electrode, the injection needle being in fluid tight engagement with the lumen of the tip electrode, wherein the injection needle is movable between a first position in which the needle is contained within the tip electrode, to a second position in which the needle extends out of the distal face of the tip electrode; a deflection control handle proximal the catheter body, the deflection control handle having a thumb control wherein the entire thumb control is longitudinally movable relative to the deflection control handle; a needle control handle proximal the deflection control handle and connected to the injection needle for moving the injection needle longitudinally relative to the catheter body.

Example 25: The method of any one of Examples 15-18, wherein the injection catheter comprises: a catheter body comprising an outer wall, proximal and distal ends and at least one lumen extending therethrough; a tip section comprising flexible tubing having proximal and distal ends and at least one lumen having an inner diameter extending therethrough, the proximal end of the tip section being fixedly attached to the distal end of the catheter body; an injection needle having proximal and distal ends and an outer diameter extending through the at least one lumen in the catheter body, then extending through the at least one lumen of the tip section, the injection needle being in fluid tight engagement with the at least one lumen of the tip section through which it extends, wherein the injection needle is slidable between a retracted position in which the injection needle is contained within the tip section and an extended position in which the injection needle extends out of the distal end of the tip section; a deflection control handle having proximal and distal ends mounted on the proximal end of the catheter body; a thumb control, wherein the entire thumb control is longitudinally moveable relative to the deflection control handle, the thumb control being mounted on the deflection control handle; a first puller wire having proximal and distal ends extending through the deflection control handle and the catheter body, the proximal end of the puller wire being anchored in the deflection control handle and the distal end of the puller wire being anchored to the tip section; and a needle control handle having a piston.

Example 26: The method of any one of Examples 15-18, wherein the injection catheter comprises: a catheter body comprising an outer wall, proximal and distal ends and at least one lumen extending therethrough; a tip section comprising flexible tubing and having proximal and distal ends and at least one lumen extending therethrough, the proximal end of the tip section being fixedly attached to the distal end of the catheter body; a tip electrode mounted on the distal end of the tip section, the tip electrode having a distal face and a lumen extending therethrough; an injection needle having proximal and distal ends extending through the at least one lumen of the tip section into the lumen of the tip electrode, the injection needle being in fluid tight engagement with the lumen of the tip electrode, wherein the injection needle is slidable between a retracted position in which the injection needle is contained within the tip section and an extended position in which the injection needle extends out of the distal face of the tip electrode; a deflection control handle having a thumb control, the entire thumb control being longitudinally slidable relative to the deflection control handle; a puller wire having proximal and distal ends extending through the deflection control handle and the catheter body, the proximal end of the puller wire being anchored in the deflection control handle, and the distal end of the puller wire being anchored to the tip section; and a needle control handle having a piston.

Example 27: The method of any one of Examples 15-18, wherein the injection catheter comprises: a catheter body comprising a flexible tubing having proximal and distal ends and at least one lumen extending therethrough; a tip section comprising a flexible tubing having proximal and distal ends, wherein the proximal end of the tip section is mounted at the distal end of the catheter body; a tip electrode mounted on the distal end of the tip section; a needle control handle at the proximal end of the catheter body; an injection needle comprising elongated tubing extending through the catheter body, tip section and needle control handle and having a proximal end attached to the needle control handle, the injection needle also having a distal region within the tip section and an open distal end, wherein the injection needle is longitudinally slidable within the catheter body so that its distal region can extend beyond the distal end of the tip section upon suitable manipulation of the needle control handle, and further wherein the distal region of the injection needle has four fluid ports along its length in addition to the open distal end of the needle, wherein a first pair of fluid ports is positioned on one side of the injection needle and a second-pair of fluid ports is positioned on an opposite side of the injection needle, wherein each pair of fluid ports includes a distal-most fluid port and a proximal-most fluid port, the distal-most fluid port of each pair of fluid ports being positioned about two inches from the distal end of the injection needle, and the proximal-most fluid port of each pair of fluid ports being distanced about 0.02 inches from its corresponding distal-most fluid port; a protective tube mounted coaxially around the injection needle; and an electromagnetic location sensor mounted in the distal end of the tip section; whereby, in use, fluid passes out of the needle through the open distal end of the needle and through the fluid ports along the length of the distal region.

Example 28: The method of any one of Examples 15-18, wherein the injection catheter comprises: a catheter body comprising an outer wall, proximal and distal ends and at least one lumen extending therethrough; a tip section comprising a flexible tubing having proximal and distal ends, wherein the proximal end of the tip section is mounted at the distal end of the catheter body; a needle control handle at the proximal end of the catheter body; an injection needle comprising elongated tubing extending through the tip section, catheter body, and needle control handle and having a proximal end attached to the needle control handle, the injection needle also having a distal region within the tip section and an open distal end, wherein the injection needle is longitudinally slidable within the tip section so that its distal region can extend beyond the distal end of the tip section upon suitable manipulation of the needle control handle, and further wherein the distal region of the injection needle has four fluid ports along its length in addition to the open distal end of the needle, wherein a first pair of fluid ports is positioned on one side of the injection needle and a second-pair of fluid ports is positioned on an opposite side of the injection needle, wherein each pair of fluid ports includes a distal-most fluid port and a proximal-most fluid port; a protective tube mounted coaxially around the injection needle; whereby, in use, fluid passes out of the needle through the open distal end of the needle and through the fluid ports along the length of the distal region.

Example 29: The method of any one of Examples 15-18, wherein the injection catheter comprises: a catheter body comprising flexible tubing having proximal and distal ends, a stiffening tube disposed within the flexible tubing, and at least one lumen extending therethrough; a needle control handle at the proximal end of the catheter body, the needle control handle comprising: an outer body having a piston chamber therein; a piston slidably mounted within the piston chamber; and a compression spring in the piston chamber between the outer body and the piston; an injection needle extending through the catheter body and at least a portion of the needle control handle and having a proximal end attached to one of the outer body and the piston and a distal end within the catheter body; whereby longitudinal movement of one of the outer body and the piston relative to the other of the outer body and the piston compresses the compression spring and moves the injection needle distally relative to the catheter body so that the distal end of the injection needle extends outside the distal end of the catheter body.

Example 30: The method of any one of Examples 15-18, wherein the injection catheter comprises: a catheter body having an outer wall, proximal and distal ends, and at least one lumen extending therethrough; a control handle fixedly attached to the proximal end of the catheter body; a tip section comprising flexible tubing having proximal and distal ends and at least one lumen extending therethrough, the proximal end of the tip section being fixedly attached to the distal end of the catheter body; an injection needle which is straight and smooth over its entire length extends through the lumen in the catheter body and extending in fluid-tight engagement through the lumen in the tip section, said needle being slidable from a first position in which the needle is withdrawn into the tip section to a second position in which the needle extends out of the tip section; a slidable needle control knob mounted on the control handle and connected to the proximal end of the injection needle for sliding the injection needle from the first position to the second position; and, a deflection control mounted on the control handle for deflecting the catheter upon manipulation of the deflection control.

Example 31: The method of any one of Examples 15-18, wherein the injection catheter comprises: a catheter body; an intermediate section distal of the catheter body; a tip section distal of the intermediate section, the tip section comprising: a tip electrode with an omnidirectional distal end and a concentric needle port; and an integrated magnetic device and position sensor, at least one of which having a hollow configuration to receive at least a portion of the other; a transition section between the intermediate section and the tip section and having one or more additional magnetic devices, a needle extending through the catheter body, the intermediate section and the tip section; and a needle control handle adapted to extend and retract the injection needle through the concentric needle port.

Example 32: The method of any one of Examples 15-31, wherein the target location comprises the myocardium.

Example 33: The method of any one of Examples 15-32, wherein the target location comprises an area at or adjacent the endocardial surface of the heart.

Example 34: The method of any one of Examples 15-33, wherein the electroconductive material comprises a plurality of electrically conductive nanoparticles.

Example 35: The method of any one of Examples 15-34, wherein the electroconductive material comprises an aqueous dispersion of metallic nanoparticles.

Example 36: The method of any one of Examples 15-35, wherein the electroconductive material comprises a plurality of metallic nanoparticles.

Example 37: The method of Example 36, wherein the plurality of electrically conductive nanoparticles comprises at least one biocompatible metal such as gold, platinum, palladium, nickel, copper, zinc, or silver.

Example 38: The method of any one of Examples 34-37, wherein the average diameter of the electrically conductive nanoparticles is less than 150 nm.

Example 39: The method of any one of Examples 15-38, wherein the electroconductive material comprises a plurality of electrically conductive nanoparticles having a coating on a surface thereof, wherein the coating prevents the particles from agglomerating when the particles are in solution and allows adjacent particle surfaces to be in direct physical contact when the particles are not in solution.

Example 40: The method of any one of Examples 15-39, wherein the electroconductive material comprises a plurality of substantially non-agglomerated metal nanoparticles, wherein the plurality of substantially non-agglomerated nanoparticles has an electrical resistivity of 1 Ω-cm or less.

Example 41: The method of any one of Examples 15-40, wherein the electroconductive material comprises a plurality of electrically conductive polymer molecules, wherein the average diameter of the electrically conductive polymer molecules is less than 150 nm.

Example 42: The method of Example 41, wherein the electrically conductive polymer comprises polyvinylpyrrolidone.

Example 43: The method of any one of Examples 15-42, wherein the electroconductive material comprises a conductive choline-based bio-ionic liquid (Bio-IL) conjugated hydrogel.

Example 44: The method of any one of Examples 15-43, wherein the electroconductive material comprises a fluid suspension of carbon nanotube particles.

Example 45: The method of any one of Examples 15-44, wherein the electroconductive material comprises a fluid suspension of graphene particles.

Example 46: An injection catheter for the improvement of intramyocardial conduction delay, the catheter comprising: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a needle control handle disposed adjacent the proximal end of the catheter body; an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 47: The injection catheter of Example 46, further comprising a thumb control mounted on the needle control handle for deflecting the catheter upon manipulation of the thumb control.

Example 48: The injection catheter of any one of Examples 46-47, further comprising one or more position sensors disposed in or adjacent the tip section and configured to determine a position of the tip section.

Example 49: The injection catheter of any one of Examples 46-48, further comprising one or more position sensors disposed in or adjacent the tip section and configured to determine an orientation of the tip section.

Example 50: The injection catheter of any one of Examples 46-49, further comprising one or more contact sensors disposed in or adjacent the tip section and configured to determine contact of the tip section to a surface.

Example 51: The injection catheter of any one of Examples 46-50, wherein the distal end of the tip section has an omnidirectional configuration.

Example 52: The injection catheter of any one of Examples 46-51, wherein the distal end of the tip section has a dome shape.

Example 53: The injection catheter of any one of Examples 46-52, wherein a needle port is disposed on the distal end of the tip section and is configured to allow the needle to extend outside the distal end of the tip section.

Example 54: The catheter of Example 53, wherein the needle port is disposed concentrically to the distal end of the tip section.

Example 55: The injection catheter of any one of Examples 46-54, wherein the catheter body comprises an intermediate section disposed between the tip section and the needle control handle.

Example 56: The catheter of Example 55, wherein the intermediate section comprises a low durometer tubing reinforced with a spring coil.

Example 57: An injection catheter for the improvement of intramyocardial conduction delay, the catheter comprising: a catheter body comprising a flexible tubing having proximal and distal ends and at least one lumen extending therethrough; a tip section disposed adjacent the distal end of the catheter body; a needle control handle disposed adjacent the proximal end of the catheter body; an injection needle disposed in at least a portion of the tip section, at least a portion of the catheter body, and at least a portion the needle control handle, the injection needle having a proximal end coupled to the needle control handle, wherein the injection needle is longitudinally slidable so that the distal end of the injection needle extends beyond the distal end of the tip section upon suitable manipulation of the needle control handle; and a fluid reservoir in fluid-communication with the injection needle and configured for the delivery of electroconductive material through the injection needle to a target location.

What is claimed is:

1. A method for improvement of intramyocardial conduction delay, the method comprising:
guiding an injection catheter to a target location in a body of a patient;
causing an injection needle to deploy; and
causing, via the deployed injection needle, delivery of a solution comprising biocompatible, electroconductive material to the target location.

2. The method of claim 1, further comprising:
positioning the injection catheter proximate an injection site within the target location; and
advancing the injection needle into tissue at the injection site,
wherein the causing delivery comprises delivering the electroconductive material to the injection site within the target location.

3. The method of claim 1, wherein the injection catheter comprises:
a catheter body comprising a flexible tubing having a proximal end and a distal end and at least one lumen disposed through at least a portion of the flexible tubing;
a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter;
a needle control handle disposed adjacent the proximal end of the catheter body;
an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and
a needle stop disposed on a portion of the injection needle, wherein the needle stop has a distal end that is sized to prevent passage of the portion of the injection needle on which the needle stop is mounted from passing into the distal region of the needle passage thereby limiting a distance that the injection needle is capable of extending beyond the distal end of the tip section.

4. The method of claim 1, wherein the injection catheter comprises:
a catheter body comprising a flexible tubing having proximal and distal ends and at least one lumen extending therethrough;
a tip section disposed adjacent the distal end of the catheter body;
a needle control handle disposed adjacent the proximal end of the catheter body;
an injection needle disposed in at least a portion of the tip section, at least a portion of the catheter body, and at least a portion of the needle control handle, the injection needle having a proximal end coupled to the needle control handle, wherein the injection needle is longitudinally slidable so that the distal end of the injection needle extends beyond the distal end of the tip section upon suitable manipulation of the needle control handle; and
a fluid reservoir in fluid-communication with the injection needle and configured for delivery of electroconductive material through the injection needle to a target location.

5. The method of claim 1, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
one or more electrodes mounted on the tip section;
one or more electrode lead wires that correspond in number to the number of electrodes, each electrode lead wire having a distal end that is electrically connected to a corresponding electrode on the tip section;
a needle control handle at the proximal end of the catheter body;
an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle consists of plastic tubing having at least a distal region with straight position memory; and
a protective tubing extending through the tip section and catheter body through which the needle extends coaxially.

6. The method of claim 1, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
one or more electrodes mounted on the tip section;
one or more electrode lead wires that correspond in number to the number of electrodes, each electrode lead wire having a distal end that is electrically connected to a corresponding electrode on the tip section;
a needle control handle proximal the catheter body;
an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle comprises plastic tubing having at least a distal region with straight position memory, the injection needle further comprising a piece of metal tubing attached at its end to an end of the plastic tubing; and
a protective tubing through which the needle extends coaxially.

7. The method of claim 1, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
one or more electrodes mounted on the tip section;
one or more electrode lead wires that correspond in number to the number of electrodes, each electrode lead wire having a distal end that is electrically connected to a corresponding electrode on the tip section;
a needle control handle proximal the catheter body;
an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle comprises plastic tubing having at least a distal region with straight position memory;
a protective tubing through which the needle extends coaxially; and
a deflection control handle proximal of the catheter body and distal of the needle control handle, wherein the injection needle and protective tube extend through a guide tube thereby allowing the injection needle and protective tube longitudinal movement within the deflection control handle, the deflection control handle having a piston for manipulating a puller wire, wherein the injection needle, protective tube and guide tube extend through a second tunnel in the deflection control handle and a space is provided between a proximal end of the piston and a distal end of the second tunnel to avoid undesirable bending of the injection needle.

8. The method of claim 1, wherein the injection catheter comprises:
a catheter body having an outer wall, proximal and distal ends, and at least one lumen extending therethrough;
a tip section comprising flexible tubing and having proximal and distal ends and at least one lumen extending therethrough, the proximal end of the tip section being fixedly attached to the distal end of the catheter body;
a tip electrode mounted at the distal end of the tip section, the tip electrode having a distal face and a lumen extending therethrough;
an injection needle which is straight and smooth over its entire length extending through the at least one lumen of the catheter body, then extending through the at least one lumen of the tip section and into the lumen of the tip electrode, the injection needle being in fluid tight engagement with the lumen of the tip electrode, wherein the injection needle is movable between a first position in which the needle is contained within the tip electrode, to a second position in which the needle extends out of the distal face of the tip electrode;
a deflection control handle proximal the catheter body, the deflection control handle having a thumb control wherein the thumb control is longitudinally movable relative to the deflection control handle;
a needle control handle proximal the deflection control handle and connected to the injection needle for moving the injection needle longitudinally relative to the catheter body.

9. The method of claim 1, wherein the injection catheter comprises:
a catheter body comprising an outer wall, proximal and distal ends and at least one lumen extending therethrough;
a tip section comprising flexible tubing having proximal and distal ends and at least one lumen having an inner diameter extending therethrough, the proximal end of the tip section being fixedly attached to the distal end of the catheter body;
an injection needle having proximal and distal ends and an outer diameter extending through the at least one lumen in the catheter body, then extending through the at least one lumen of the tip section, the injection needle being in fluid tight engagement with the at least one lumen of the tip section through which it extends, wherein the injection needle is slidable between a retracted position in which the injection needle is contained within the tip section and an extended position in which the injection needle extends out of the distal end of the tip section;
a deflection control handle having proximal and distal ends mounted on the proximal end of the catheter body;
a thumb control, wherein the thumb control is longitudinally moveable relative to the deflection control handle, the thumb control being mounted on the deflection control handle;
a first puller wire having proximal and distal ends extending through the deflection control handle and the catheter body, the proximal end of the puller wire being anchored in the deflection control handle and the distal end of the puller wire being anchored to the tip section; and
a needle control handle having a piston.

10. The method of claim 1, wherein the injection catheter comprises:
a catheter body comprising an outer wall, proximal and distal ends and at least one lumen extending therethrough;
a tip section comprising flexible tubing and having proximal and distal ends and at least one lumen extending therethrough, the proximal end of the tip section being fixedly attached to the distal end of the catheter body;
a tip electrode mounted on the distal end of the tip section, the tip electrode having a distal face and a lumen extending therethrough;
an injection needle having proximal and distal ends extending through the at least one lumen of the tip section into the lumen of the tip electrode, the injection needle being in fluid tight engagement with the lumen of the tip electrode, wherein the injection needle is slidable between a retracted position in which the injection needle is contained within the tip section and an extended position in which the injection needle extends out of the distal face of the tip electrode;
a deflection control handle having a thumb control, the thumb control being longitudinally slidable relative to the deflection control handle;
a puller wire having proximal and distal ends extending through the deflection control handle and the catheter body, the proximal end of the puller wire being anchored in the deflection control handle, and the distal end of the puller wire being anchored to the tip section; and
a needle control handle having a piston.

11. The method of claim 1, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
a tip electrode mounted on the distal end of the tip section;
a needle control handle at the proximal end of the catheter body;
an injection needle comprising elongated tubing extending through the catheter body, tip section and needle control handle and having a proximal end attached to the needle control handle, the injection needle also having a distal region within the tip section and an open distal end, wherein the injection needle is longitudinally slidable within the catheter body so that its distal region can extend beyond the distal end of the tip section upon suitable manipulation of the needle control handle, and further wherein the distal region of the injection needle has four fluid ports along its length in addition to the open distal end of the needle, wherein a first pair of fluid ports is positioned on one side of the injection needle and a second-pair of fluid ports is positioned on an opposite side of the injection needle, wherein each pair of fluid ports includes a distal-most fluid port and a proximal-most fluid port, the distal-most fluid port of each pair of fluid ports being positioned about two inches from the distal end of the injection needle, and the proximal-most fluid port of each pair of fluid ports being distanced about 0.02 inches from its corresponding distal-most fluid port;
a protective tube mounted coaxially around the injection needle; and
an electromagnetic location sensor mounted in the distal end of the tip section;
whereby, in use, fluid passes out of the needle through the open distal end of the needle and through the fluid ports along the length of the distal region.

12. The method of claim 1, wherein the injection catheter comprises:
a catheter body comprising an outer wall, proximal and distal ends and at least one lumen extending therethrough;
a tip section comprising a flexible tubing having proximal and distal ends, wherein the proximal end of the tip section is mounted at the distal end of the catheter body;
a needle control handle at the proximal end of the catheter body;
an injection needle comprising elongated tubing extending through the tip section, catheter body, and needle control handle and having a proximal end attached to the needle control handle, the injection needle also having a distal region within the tip section and an open distal end, wherein the injection needle is longitudinally slidable within the tip section so that its distal region can extend beyond the distal end of the tip section upon suitable manipulation of the needle control handle, and further wherein the distal region of the injection needle has four fluid ports along its length in addition to the open distal end of the needle, wherein a first pair of fluid ports is positioned on one side of the injection needle and a second-pair of fluid ports is positioned on an opposite side of the injection needle, wherein each pair of fluid ports includes a distal-most fluid port and a proximal-most fluid port;
a protective tube mounted coaxially around the injection needle;
whereby, in use, fluid passes out of the needle through the open distal end of the needle and through the fluid ports along the length of the distal region.

13. The method of claim 1, wherein the injection catheter comprises:
a catheter body comprising flexible tubing having proximal and distal ends, a stiffening tube disposed within the flexible tubing, and at least one lumen extending therethrough;
a needle control handle at the proximal end of the catheter body, the needle control handle comprising:
an outer body having a piston chamber therein;
a piston slidably mounted within the piston chamber; and
a compression spring in the piston chamber between the outer body and the piston;
an injection needle extending through the catheter body and at least a portion of the needle control handle and having a proximal end attached to one of the outer body and the piston and a distal end within the catheter body;
whereby longitudinal movement of one of the outer body and the piston relative to the other of the outer body and the piston compresses the compression spring and moves the injection needle distally relative to the catheter body so that the distal end of the injection needle extends outside the distal end of the catheter body.

14. The method of claim 1, wherein the injection catheter comprises:
a catheter body having an outer wall, proximal and distal ends, and at least one lumen extending therethrough;
a control handle fixedly attached to the proximal end of the catheter body;
a tip section comprising flexible tubing having proximal and distal ends and at least one lumen extending therethrough, the proximal end of the tip section being fixedly attached to the distal end of the catheter body;
an injection needle which is straight and smooth over its entire length extends through the lumen in the catheter body and extending in fluid-tight engagement through the lumen in the tip section, said needle being slidable from a first position in which the needle is withdrawn into the tip section to a second position in which the needle extends out of the tip section;
a slidable needle control knob mounted on the control handle and connected to the proximal end of the injection needle for sliding the injection needle from the first position to the second position; and,
a deflection control mounted on the control handle for deflecting the catheter upon manipulation of the deflection control.

15. The method of claim 1, wherein the injection catheter comprises:
a catheter body;
an intermediate section distal of the catheter body;
a tip section distal of the intermediate section, the tip section comprising:
a tip electrode with an omnidirectional distal end and a concentric needle port; and
an integrated magnetic device and position sensor, at least one of which having a hollow configuration to receive at least a portion of the other;
a transition section between the intermediate section and the tip section and having one or more additional magnetic devices.
a needle extending through the catheter body, the intermediate section and the tip section; and
a needle control handle adapted to extend and retract the injection needle through the concentric needle port.

16. The method of claim 1, wherein the target location comprises the myocardium.

17. The method of claim 1, wherein the target location comprises an area at or adjacent the endocardial surface of the heart.

18. The method of claim 1, wherein the electroconductive material comprises a plurality of electrically conductive nanoparticles.

19. The method of claim 1, wherein the electroconductive material comprises an aqueous dispersion of metallic nanoparticles.

20. The method of claim 1, wherein the electroconductive material comprises a plurality of metallic nanoparticles.

21. The method of claim 18, wherein the plurality of electrically conductive nanoparticles comprises at least one biocompatible metal such as gold, platinum, palladium, nickel, copper, zinc, or silver.

22. The method of claim 18, wherein the average diameter of the electrically conductive nanoparticles is less than 150 nm.

23. The method of claim 1, wherein the electroconductive material comprises a plurality of electrically conductive nanoparticles having a coating on a surface thereof, wherein the coating prevents the particles from agglomerating when the particles are in solution and allows adjacent particle surfaces to be in direct physical contact when the particles are not in solution.

24. The method of claim 1, wherein the electroconductive material comprises a plurality of substantially non-agglomerated metal nanoparticles, wherein the plurality of substantially non-agglomerated nanoparticles has an electrical resistivity of 1 Ω-cm or less.

25. The method of claim 1, wherein the electroconductive material comprises a plurality of electrically conductive polymer molecules, wherein the average diameter of the electrically conductive polymer molecules is less than 150 nm.

26. The method of claim 25, wherein the electrically conductive polymer comprises polyvinylpyrrolidone.

27. The method of claim 1, wherein the electroconductive material comprises a conductive choline-based bio-ionic liquid (Bio-IL) conjugated hydrogel.

28. The method of claim 1, wherein the electroconductive material comprises a fluid suspension of carbon nanotube particles.

29. The method of claim 1, wherein the electroconductive material comprises a fluid suspension of graphene particles.

30. A method for improvement of intramyocardial conduction delay, the method comprising:
guiding an injection catheter to a target location in a body of a patient;
positioning the injection catheter proximate a first injection site;
causing an injection needle to deploy;
advancing the injection needle into tissue at the first injection site;
delivering an electroconductive material to the first injection site;
causing an injection needle to retract;
positioning the injection catheter proximate at least one other injection site located a desired distance away from the first injection site;
causing an injection needle to deploy;
advancing the injection needle into tissue at least one other injection site located a desired distance away from the first injection site; and
delivering an electroconductive material to at least one other injection site located a desired distance away from the first injection site.

31. The method of claim 30, wherein the injection catheter comprises:
a catheter body comprising a flexible tubing having a proximal end and a distal end and at least one lumen disposed through at least a portion of the flexible tubing;
a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter;
a needle control handle disposed adjacent the proximal end of the catheter body;
an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and
a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

32. The method of claim 30, wherein the injection catheter comprises:
a catheter body comprising a flexible tubing having proximal and distal ends and at least one lumen extending therethrough;
a tip section disposed adjacent the distal end of the catheter body;
a needle control handle disposed adjacent the proximal end of the catheter body;
an injection needle disposed in at least a portion of the tip section, at least a portion of the catheter body, and at least a portion of the needle control handle, the injection needle having a proximal end coupled to the needle control handle, wherein the injection needle is longitudinally slidable so that the distal end of the injection needle extends beyond the distal end of the tip section upon suitable manipulation of the needle control handle; and
a fluid reservoir in fluid-communication with the injection needle and configured for delivery of electroconductive material through the injection needle to a target location.

33. The method of claim 30, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
one or more electrodes mounted on the tip section;
one or more electrode lead wires that correspond in number to the number of electrodes, each electrode lead wire having a distal end that is electrically connected to a corresponding electrode on the tip section;
a needle control handle at the proximal end of the catheter body;
an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle consists of plastic tubing having at least a distal region with straight position memory; and
a protective tubing extending through the tip section and catheter body through which the needle extends coaxially.

34. The method of claim 30, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
one or more electrodes mounted on the tip section;
one or more electrode lead wires that correspond in number to the number of electrodes, each electrode lead wire having a distal end that is electrically connected to a corresponding electrode on the tip section;
a needle control handle proximal the catheter body;
an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle comprises plastic tubing having at least a distal region with straight position memory, the injection needle further comprising a piece of metal tubing attached at its end to an end of the plastic tubing; and
a protective tubing through which the needle extends coaxially.

35. The method of claim 30, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
one or more electrodes mounted on the tip section;
one or more electrode lead wires that correspond in number to the number of electrodes, each electrode lead wire having a distal end that is electrically connected to a corresponding electrode on the tip section;
a needle control handle proximal the catheter body;
an injection needle extending through the tip section, catheter body, and needle control handle and having a proximal end attached to the needle control handle and a distal end within the tip section, wherein the injection needle is longitudinally slidable within the tip section so that the distal end of the injection needle can extend beyond the distal end of the tip section upon suitable manipulation of the needle control handle, and further wherein the injection needle comprises plastic tubing having at least a distal region with straight position memory;
a protective tubing through which the needle extends coaxially; and
a deflection control handle proximal of the catheter body and distal of the needle control handle, wherein the injection needle and protective tube extend through a guide tube thereby allowing the injection needle and protective tube longitudinal movement within the deflection control handle, the deflection control handle having a piston for manipulating a puller wire, wherein the injection needle, protective tube and guide tube extend through a second tunnel in the deflection control handle and a space is provided between a proximal end of the piston and a distal end of the second tunnel to avoid undesirable bending of the injection needle.

36. The method of claim 30, wherein the injection catheter comprises:
a catheter body having an outer wall, proximal and distal ends, and at least one lumen extending therethrough;
a tip section comprising flexible tubing and having proximal and distal ends and at least one lumen extending therethrough, the proximal end of the tip section being fixedly attached to the distal end of the catheter body;
a tip electrode mounted at the distal end of the tip section, the tip electrode having a distal face and a lumen extending therethrough;
an injection needle which is straight and smooth over its entire length extending through the at least one lumen of the catheter body, then extending through the at least one lumen of the tip section and into the lumen of the tip electrode, the injection needle being in fluid tight engagement with the lumen of the tip electrode, wherein the injection needle is movable between a first position in which the needle is contained within the tip electrode, to a second position in which the needle extends out of the distal face of the tip electrode;
a deflection control handle proximal the catheter body, the deflection control handle having a thumb control wherein the thumb control is longitudinally movable relative to the deflection control handle;
a needle control handle proximal the deflection control handle and connected to the injection needle for moving the injection needle longitudinally relative to the catheter body.

37. The method of claim 30, wherein the injection catheter comprises:
a catheter body comprising an outer wall, proximal and distal ends and at least one lumen extending therethrough;
a tip section comprising flexible tubing having proximal and distal ends and at least one lumen having an inner diameter extending therethrough, the proximal end of the tip section being fixedly attached to the distal end of the catheter body;
an injection needle having proximal and distal ends and an outer diameter extending through the at least one lumen in the catheter body, then extending through the at least one lumen of the tip section, the injection needle being in fluid tight engagement with the at least one lumen of the tip section through which it extends, wherein the injection needle is slidable between a retracted position in which the injection needle is contained within the tip section and an extended position in which the injection needle extends out of the distal end of the tip section;
a deflection control handle having proximal and distal ends mounted on the proximal end of the catheter body;
a thumb control, wherein the thumb control is longitudinally moveable relative to the deflection control handle, the thumb control being mounted on the deflection control handle;
a first puller wire having proximal and distal ends extending through the deflection control handle and the catheter body, the proximal end of the puller wire being anchored in the deflection control handle and the distal end of the puller wire being anchored to the tip section; and
a needle control handle having a piston.

38. The method of claim 30, wherein the injection catheter comprises:
a catheter body comprising an outer wall, proximal and distal ends and at least one lumen extending therethrough;
a tip section comprising flexible tubing and having proximal and distal ends and at least one lumen extending therethrough, the proximal end of the tip section being fixedly attached to the distal end of the catheter body;
a tip electrode mounted on the distal end of the tip section, the tip electrode having a distal face and a lumen extending therethrough;
an injection needle having proximal and distal ends extending through the at least one lumen of the tip section into the lumen of the tip electrode, the injection needle being in fluid tight engagement with the lumen of the tip electrode, wherein the injection needle is slidable between a retracted position in which the injection needle is contained within the tip section and an extended position in which the injection needle extends out of the distal face of the tip electrode;
a deflection control handle having a thumb control, the thumb control being longitudinally slidable relative to the deflection control handle;
a puller wire having proximal and distal ends extending through the deflection control handle and the catheter body, the proximal end of the puller wire being anchored in the deflection control handle, and the distal end of the puller wire being anchored to the tip section; and
a needle control handle having a piston.

39. The method of claim 30, wherein the injection catheter comprises:
a catheter body comprising a flexible tubing having proximal and distal ends and at least one lumen extending therethrough;
a tip section comprising a flexible tubing having proximal and distal ends, wherein the proximal end of the tip section is mounted at the distal end of the catheter body;
a tip electrode mounted on the distal end of the tip section;
a needle control handle at the proximal end of the catheter body;
an injection needle comprising elongated tubing extending through the catheter body, tip section and needle control handle and having a proximal end attached to the needle control handle, the injection needle also having a distal region within the tip section and an open distal end, wherein the injection needle is longitudinally slidable within the catheter body so that its distal region can extend beyond the distal end of the tip section upon suitable manipulation of the needle control handle, and further wherein the distal region of the injection needle has four fluid ports along its length in addition to the open distal end of the needle, wherein a first pair of fluid ports is positioned on one side of the injection needle and a second-pair of fluid ports is positioned on an opposite side of the injection needle, wherein each pair of fluid ports includes a distal-most fluid port and a proximal-most fluid port, the distal-most fluid port of each pair of fluid ports being positioned about two inches from the distal end of the injection needle, and the proximal-most fluid port of each pair of fluid ports being distanced about 0.02 inches from its corresponding distal-most fluid port;
a protective tube mounted coaxially around the injection needle; and
an electromagnetic location sensor mounted in the distal end of the tip section;
whereby, in use, fluid passes out of the needle through the open distal end of the needle and through the fluid ports along the length of the distal region.

40. The method of claim 30, wherein the injection catheter comprises:
a catheter body comprising an outer wall, proximal and distal ends and at least one lumen extending therethrough;
a tip section comprising a flexible tubing having proximal and distal ends, wherein the proximal end of the tip section is mounted at the distal end of the catheter body;
a needle control handle at the proximal end of the catheter body;
an injection needle comprising elongated tubing extending through the tip section, catheter body, and needle control handle and having a proximal end attached to the needle control handle, the injection needle also having a distal region within the tip section and an open distal end, wherein the injection needle is longitudinally slidable within the tip section so that its distal region can extend beyond the distal end of the tip section upon suitable manipulation of the needle control handle, and further wherein the distal region of the injection needle has four fluid ports along its length in addition to the open distal end of the needle, wherein a first pair of fluid ports is positioned on one side of the injection needle and a second-pair of fluid ports is positioned on an opposite side of the injection needle, wherein each pair of fluid ports includes a distal-most fluid port and a proximal-most fluid port;
a protective tube mounted coaxially around the injection needle;
whereby, in use, fluid passes out of the needle through the open distal end of the needle and through the fluid ports along the length of the distal region.

41. The method of claim 30, wherein the injection catheter comprises:
a catheter body comprising flexible tubing having proximal and distal ends, a stiffening tube disposed within the flexible tubing, and at least one lumen extending therethrough;
a needle control handle at the proximal end of the catheter body, the needle control handle comprising:
an outer body having a piston chamber therein;
a piston slidably mounted within the piston chamber; and
a compression spring in the piston chamber between the outer body and the piston;
an injection needle extending through the catheter body and at least a portion of the needle control handle and having a proximal end attached to one of the outer body and the piston and a distal end within the catheter body;
whereby longitudinal movement of one of the outer body and the piston relative to the other of the outer body and the piston compresses the compression spring and moves the injection needle distally relative to the catheter body so that the distal end of the injection needle extends outside the distal end of the catheter body.

42. The method of claim 30, wherein the injection catheter comprises:
a catheter body having an outer wall, proximal and distal ends, and at least one lumen extending therethrough;
a control handle fixedly attached to the proximal end of the catheter body;
a tip section comprising flexible tubing having proximal and distal ends and at least one lumen extending therethrough, the proximal end of the tip section being fixedly attached to the distal end of the catheter body;
an injection needle which is straight and smooth over its entire length extends through the lumen in the catheter body and extending in fluid-tight engagement through the lumen in the tip section, said needle being slidable from a first position in which the needle is withdrawn into the tip section to a second position in which the needle extends out of the tip section;
a slidable needle control knob mounted on the control handle and connected to the proximal end of the injection needle for sliding the injection needle from the first position to the second position; and,
a deflection control mounted on the control handle for deflecting the catheter upon manipulation of the deflection control.

43. The method of claim 30, wherein the injection catheter comprises:
a catheter body;
an intermediate section distal of the catheter body;
a tip section distal of the intermediate section, the tip section comprising:
a tip electrode with an omnidirectional distal end and a concentric needle port; and
an integrated magnetic device and position sensor, at least one of which having a hollow configuration to receive at least a portion of the other;
a transition section between the intermediate section and the tip section and having one or more additional magnetic devices.
a needle extending through the catheter body, the intermediate section and the tip section; and
a needle control handle adapted to extend and retract the injection needle through the concentric needle port.

44. The method of claim 30, wherein the target location comprises the myocardium.

45. The method of claim 30, wherein the target location comprises an area at or adjacent the endocardial surface of the heart.

46. The method of claim 30, wherein the electroconductive material comprises a plurality of electrically conductive nanoparticles.

47. The method of claim 30, wherein the electroconductive material comprises an aqueous dispersion of metallic nanoparticles.

48. The method of claim 30, wherein the electroconductive material comprises a plurality of metallic nanoparticles.

49. The method of claim 46, wherein the plurality of electrically conductive nanoparticles comprises at least one biocompatible metal such as gold, platinum, palladium, nickel, copper, zinc, or silver.

50. The method of claim 46, wherein the average diameter of the electrically conductive nanoparticles is less than 150 nm.

51. The method of claim 30, wherein the electroconductive material comprises a plurality of electrically conductive nanoparticles having a coating on a surface thereof, wherein the coating prevents the particles from agglomerating when the particles are in solution and allows adjacent particle surfaces to be in direct physical contact when the particles are not in solution.

52. The method of claim 30, wherein the electroconductive material comprises a plurality of substantially non-agglomerated metal nanoparticles, wherein the plurality of substantially non-agglomerated nanoparticles has an electrical resistivity of 1 Ω-cm or less.

53. The method of claim 30, wherein the electroconductive material comprises a plurality of electrically conductive polymer molecules, wherein the average diameter of the electrically conductive polymer molecules is less than 150 nm.

54. The method of claim 53, wherein the electrically conductive polymer comprises polyvinylpyrrolidone.

55. The method of claim 30, wherein the electroconductive material comprises a conductive choline-based bio-ionic liquid (Bio-IL) conjugated hydrogel.

56. The method of claim 30, wherein the electroconductive material comprises a fluid suspension of carbon nanotube particles.

57. The method of claim 30, wherein the electroconductive material comprises a fluid suspension of graphene particles.

58. An injection catheter for the improvement of intramyocardial conduction delay, the catheter comprising:
a catheter body comprising a flexible tubing having a proximal end and a distal end and at least one lumen disposed through at least a portion of the flexible tubing;
a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter;
a needle control handle disposed adjacent the proximal end of the catheter body;
an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and
a needle stop disposed on a portion of the injection needle, wherein the needle stop has a distal end that is sized to prevent passage of the portion of the injection needle on which the needle stop is mounted from passing into the distal region of the needle passage thereby limiting a distance that the injection needle is capable of extending beyond the distal end of the tip section.

59. The injection catheter of claim 58, further comprising a thumb control mounted on the needle control handle for deflecting the catheter upon manipulation of the thumb control.

60. The injection catheter of claim 58, further comprising one or more position sensors disposed in or adjacent the tip section and configured to determine a position of the tip section.

61. The injection catheter of claim 58, further comprising one or more position sensors disposed in or adjacent the tip section and configured to determine an orientation of the tip section.

62. The injection catheter of claim 58, further comprising one or more contact sensors disposed in or adjacent the tip section and configured to determine contact of the tip section to a surface.

63. The injection catheter of claim 58, wherein the distal end of the tip section has an omnidirectional configuration.

64. The injection catheter of claim 58, wherein the distal end of the tip section has a dome shape.

65. The injection catheter of claim 58, wherein a needle port is disposed on the distal end of the tip section and is configured to allow the needle to extend outside the distal end of the tip section.

66. The catheter of claim 65, wherein the needle port is disposed concentrically to the distal end of the tip section.

67. The injection catheter of claim 58, wherein the catheter body comprises an intermediate section disposed between the tip section and the needle control handle.

68. The catheter of claim 67, wherein the intermediate section comprises a low durometer tubing reinforced with a spring coil.

69. An injection catheter for the improvement of intramyocardial conduction delay, the catheter comprising:
a catheter body comprising a flexible tubing having proximal and distal ends and at least one lumen extending therethrough;
a tip section disposed adjacent the distal end of the catheter body;
a needle control handle disposed adjacent the proximal end of the catheter body;
an injection needle disposed in at least a portion of the tip section, at least a portion of the catheter body, and at least a portion the needle control handle, the injection needle having a proximal end coupled to the needle control handle, wherein the injection needle is longitudinally slidable so that the distal end of the injection needle extends beyond the distal end of the tip section upon suitable manipulation of the needle control handle; and
a fluid reservoir in fluid-communication with the injection needle and configured for delivery of electroconductive material through the injection needle to a target location.
